(19) United States
(12) Patent Application Publication
(10) Pub. No.: US 2014/0088627 A1
(43) Pub. Date: Mar. 27, 2014

(54) TONGUE CLEANING APPARATUS

(71) Applicant: Thomas Hautaniemi, Aitkenvale (AU)

(72) Inventor: Thomas Hautaniemi, Aitkenvale (AU)

(21) Appl. No.: 14/033,812

(22) Filed: Sep. 23, 2013

(30) Foreign Application Priority Data
Sep. 21, 2012 (AU) 2012904143

(51) Int. Cl. A61B 17/24

(52) U.S. Cl. CPC A61B 17/244
USPC 606/161

(57) ABSTRACT

A tongue cleaning apparatus including at least one positioning body moulded to fit over at least some teeth of a user and associated with at least one portion having with a plurality of shaped protrusions protruding from at least one external surface thereof and a method of using the tongue cleaning apparatus is provided.

### CROSS-REFERENCE TO RELATED APPLICATIONS

This application claims the benefit of Australian Patent Application No. 2012904143 filed on Sep. 21, 2012. All disclosures of the document(s) named above are incorporated herein by reference.

### BACKGROUND OF THE INVENTION

1. Field of the Invention

The present invention relates to oral hygiene generally and to a tongue cleaning apparatus in particular.

2. Description of the Related Art

A variety of tongue cleaning apparatus are conventionally available.

A tongue cleaner (also called a tongue scraper or tongue brush) is an oral hygiene device designed to clean the bacterial build-up, food debris, fungi, and dead cells from the surface of the tongue. The bacteria and fungi that grow on the tongue are related to many common oral care and general health problems. In addition, decaying bacteria produce volatile sulphur compounds on the rear of the tongue; these molecules account for 80 to 95 percent of all cases of halitosis (bad breath).

The top surface of the tongue can be cleaned using a tongue cleaner, a tongue brush/scraper or a toothbrush. However, toothbrushes are not considered as effective for this purpose because they are designed for brushing the teeth, which have a solid structure unlike the spongy tissue of the tongue.

There is also the issue that this type of tongue cleaner can trigger the “gag reflex” if touching certain sensitive areas. Advice for using this type of tongue cleaner typically includes instruction to relax the tongue and the muscles of the throat and/or exhaling during use.

Using an inappropriately designed tongue cleaner too much or in an aggressive way may cause damage to the taste buds present on the surface of the tongue. There are claims that even in such cases, the taste buds actually regenerate soon after being damaged. Using an alternative tongue cleaner carefully may avoid such circumstances

Ergonomic tongue cleaners are shaped in accordance with the anatomy of the tongue, and are optimized to lift and trap the plaque coating and effectively clean the surface of the tongue. There are many different types of tongue cleaners; they are made from plastic, metal or other materials. Their effectiveness varies widely depending on the shape, dimensions, configuration, quality of the contact surfaces and materials used. In addition, tongue cleaning gels used in association with the tongue cleaners as anti-bacterial agents may enhance cleaning effects.

Hand held tongue cleaners are typically shaped similarly to a toothbrush with an elongate handle it to be held by user with a shaped portion that the opposite extremity. The shape portion generally includes one or more ridges or bars in order to be scraped or drawn across the tongue

It will be clearly understood that, if a prior art publication is referred to herein, this reference does not constitute an admission that the publication forms part of the common general knowledge in the art in Australia or in any other country.

### SUMMARY OF THE INVENTION

The present invention is directed to a tongue cleaning apparatus and method of using same, which may at least partially overcome at least one of the abovementioned disadvantages or provide the consumer with a useful or commercial choice.

With the foregoing in view, the present invention in one form, resides broadly in a tongue cleaning apparatus including at least one positioning body moulded to fit over at least some teeth of a user and associated with at least one portion having with a plurality of shaped protrusions protruding from at least one external surface thereof.

In an alternative form, the present invention resides in a method for using a tongue cleaning apparatus including a positioning body moulded to fit over at least some teeth of a user and associated with at least one portion having with a plurality of shaped protrusions protruding from at least one external surface thereof, the method including the steps of fitting the body of the apparatus over at least some of teeth of a user to secure the apparatus within a user's mouth and the user moving their tongue over the apparatus to clean the user's tongue.

The tongue cleaning apparatus of the present invention allows for multidirectional scraping and cleaning of the users tongue. The apparatus allows access to all portions of the tongue including the rear of the time using the particular mouth morphology and natural movements of tongue as desired by the individual using the apparatus. This allows a user to clean areas of tongue which are difficult to reach with the conventional “toothbrush” type tongue cleaners.

The configuration of the present invention particularly reduces the triggering of the gag reflex.

The present invention also provides the largest surface area possible within the user's mouth in order to increase the speed of cleaning and also the effectiveness of the clean achieved.

The tongue cleaner of the present invention may also assist in cleaning the user's teeth if a tooth cleaning tool or paste for example is provided on one or more in areas of the tongue cleaning apparatus. In this configuration, movement of the body relative to the teeth (or vice versa) may be required in order to be effective, which may counteract the effectiveness of the apparatus as a tongue cleaning apparatus.

It could also be used for cleaning the bacterial build-up, food debris, fungi, and dead cells from other oral mucosal surfaces such as the inside of the user's cheeks and lips.

The tongue cleaning apparatus of the invention includes at least one positioning body molded to fit over at least some of the teeth of the user. The purpose of the at least one positioning body is to locate the apparatus within the user's mouth, at least temporarily, and to brace the apparatus against movement which may occur due to movement of the users tongue relative to the apparatus.

The term “molded” is not intended to limit the positioning body to a particular shape or method of manufacture or fit in but merely to indicate that the at least one positioning body will typically abut or engage at least some of the teeth of user in order to position the apparatus and maintain the apparatus in position during use against accidental dislodgement or movement but not against determined removal by the user.

Preferably, each positioning body will be located over at least some teeth of the user. The most preferred positioning body is a generally arch shaped or U-shaped body that extends over the entire dental arch of the user. Typically, the preferred body will extend from the rearmost molar on one side of a user's dental arch to the rearmost molar on the other side of the dental arch. Normally, the rear of each side of the body will be open. The preferred configuration is therefore a positioning body which is U-shaped in plan but also substantially U-shaped in section.

Preferably, the positioning body of this configuration is located or positioned relative to the upper, superior or maxillary dental arch. However, the positioning body may be positioned relative to the lower, inferior or mandibular dental arch or, positioning bodies may be provided to allow positioning relative to both arches.

The positioning body may be or include partial arch portions which may be provided either some or all of the molar and/or premolar and/or canine and/or incisor teeth of either the upper and/or lower dental arch of a user.

Typically, the preferred positioning body is not interchangeable between the upper and lower dental arches as the teeth in the respective arches are typically different sizes, however, a generic positioning body may be provided which allows use with either of the dental arches.

In a particularly preferred embodiment, each positioning body will typically include a forward or outer protective flange and a rearward or inner protective flange connected by a protective web adapted to abut or overlie the occlusal surface of at least some of the teeth in the arch. This configuration preferably defines a substantially U-shaped channel for receiving the teeth therein.

The inner and outer protective flanges may be provided in different sizes, either thickness and/or height. Typically, the forward or outer protective flange is higher than the inner or rearward flange. Preferably, the inner and outer flanges are shaped to correspond to the gum lines of a user. The flanges may have regions of different thicknesses.

According to one embodiment, a bridge portion may be provided between portions of the positioning body or between positioning bodies. Where provided, the bridge will preferably extend over the palate of the user between lateral portions of the positioning body or positioning bodies of provided on different locations of the dental arch.

The most preferred embodiment is a bridge portion provided extending between the preferred U-shaped positioning body with a rear edge of the bridge portion located forwardly of the rear of the positioning body. In this configuration, the rear edge of the bridge portion will be approximately at the hard palate/soft palate transition or approximately ⅔ of the distance from the front of the mouth towards the throat.

Preferably, the bridge portion extends from the inner/rearward flange of the positioning body.

Where provided in a particularly preferred form, the bridge portion and the positioning body form a substantially unitary “mouthguard” having an arch portion to position the apparatus within the mouth and bridge portion provided at a different level to the arch portion to extend across the palate of the user.

Any transitions between the bridge portion and positioning bodies will typically be arcuate but some transitions may have a more pronounced arcuate shape than others.

The positioning body shape may be, and typically will be, molded to suit an individual's teeth or arch pattern in order to provide more secure engagement. The molding may be custom molding in which an impression of the user's teeth is taken. Then the positioning body is formed using technology, such as vacuum forming or pressure lamination, stock or ready made or preformed shapes in various sizes with little or no adjustability, or combinations of the two such as “boil and bite” positioning bodies which are typically manufactured from a thermoplastic material manufactured in a preformed shape in various sizes that can be adapted to fit more closely to an individual's teeth and gums by heating and molding, such as by boiling, and then placing in the mouth.

The positioning body will typically be manufactured of an appropriate material for oral use. Normally, a plastic material is used and increased comfort can be provided by using rubberized or resilient plastic materials. Further, the positioning body may be formed from more than one material in order to provide the desired properties to the positioning body or at particular locations on the positioning body such as a more rigid structure in some parts with a more resilient structure in other parts.

Normally, the positioning body (and the apparatus as a whole) is unitary.

The at least one positioning body is associated with at least one portion having a plurality of shaped protrusions protruding from at least one external surface thereof. As mentioned above, cleaning protrusions may be provided on an inner surface as well but this is less preferred.

Typically, the at least one portion having the shaped protrusions will be an external surface of the positioning body itself and/or a bridge portion is provided.

However, the at least one portion having the shaped protrusions may be provided in different configurations. For example, one or more plates or similar devices may be mounted to or relative to a positioning body within the mouth for extension into the mouth cavity. Providing a portion which is spaced from the positioning body may allow will provide for more efficient or simple cleaning.

Preferably, the plurality of shaped protrusions are spread over the surface of the preferred arch shaped positioning body and/or bridge portion.

According to a preferred embodiment, different types of protrusions may be provided. According to a first type, some or all of the protrusions provided may be elongate in the form of ridges. The elongate protrusions can have various orientations and typically, different orientations will be preferred in order to provide more effective cleaning. Preferably, the elongate protrusions will extend laterally across the protective web of the arch shaped positioning portion. The elongate protrusions may be provided at an angle to one another and typically, this type of protrusion will be provided at different angles. Preferably, one or more elongate protrusions will be provided at alternating directions to adjacent elongate protrusions.

Preferably, the elongate protrusions will be provided with rounded ends. The preferred shape for the elongate protrusions is a rounded shape in cross-section also. The elongate protrusions can be provided in a variety of heights and lengths.

According to a second preferred type of protrusions, at least partially spheroidal protrusions may be provided. Typically, the spheroidal protrusions will be spread over the surface of the apparatus. Preferably, spheroidal protrusions will be provided in discrete regions, normally in groups. Spheroidal protrusions may be provided in particular areas on the apparatus or in lines.

In use, the tongue cleaning apparatus of the present invention will be positioned with in the mouth manually by user and located adjacent the appropriate dental arch, typically the upper arch. The user can then press the apparatus into place. The user then moves their tongue relative to the apparatus in multiple directions and over different portions of the apparatus in order to clean bedtime. Typically, the best cleaning may be achieved if a pattern is followed. This typically utilizes the natural movements of the user's mouth and tongue to achieve cleaning of the tongue.

Any of the features described herein can be combined in any combination with any one or more of the other features described herein within the scope of the invention.

The reference to any prior art in this specification is not, and should not be taken as an acknowledgement or any form of suggestion that the prior art forms part of the common general knowledge.

Additional aspects and/or advantages of the invention will be set forth in part in the description which follows and, in part, will be obvious from the description, or may be learned by practice of the invention.

### BRIEF DESCRIPTION OF THE DRAWINGS

Various embodiments of the invention will be described with reference to the following drawings, in which:

FIG. 1 is a view from below of a tongue cleaning apparatus according to a particularly preferred embodiment of the present invention.

FIG. 2 is an isometric view from the front of the apparatus illustrated in FIG. 1.

FIG. 3 is an elevated view from the front of the apparatus illustrated in FIG. 1.

FIG. 4 is a view from above of the apparatus illustrated in FIG. 1.

FIG. 5 is a view from the rear of the apparatus illustrated in FIG. 1.

FIG. 6 is an isometric view from the side of the apparatus illustrated in FIG. 1.

FIG. 7 is an alternative view from the side of the apparatus illustrated in FIG. 1.

FIG. 8 is an alternative view from the front of the apparatus illustrated in FIG. 1.

FIG. 9 is an isometric view from below of the apparatus illustrated in FIG. 1.

FIG. 10 is a view from below of a tongue cleaning apparatus according to an alternative embodiment of the present invention.

FIGS. 11A to 11D illustrate the tongue cleaning apparatus of the preferred embodiments of the present invention in use.

### DETAILED DESCRIPTION OF THE EMBODIMENTS

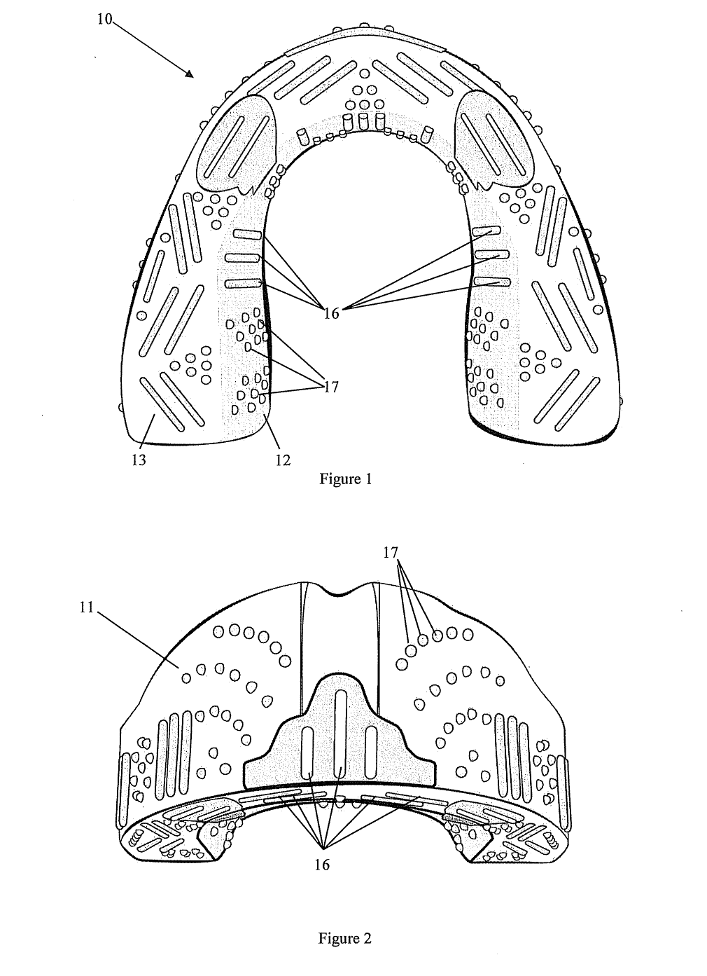

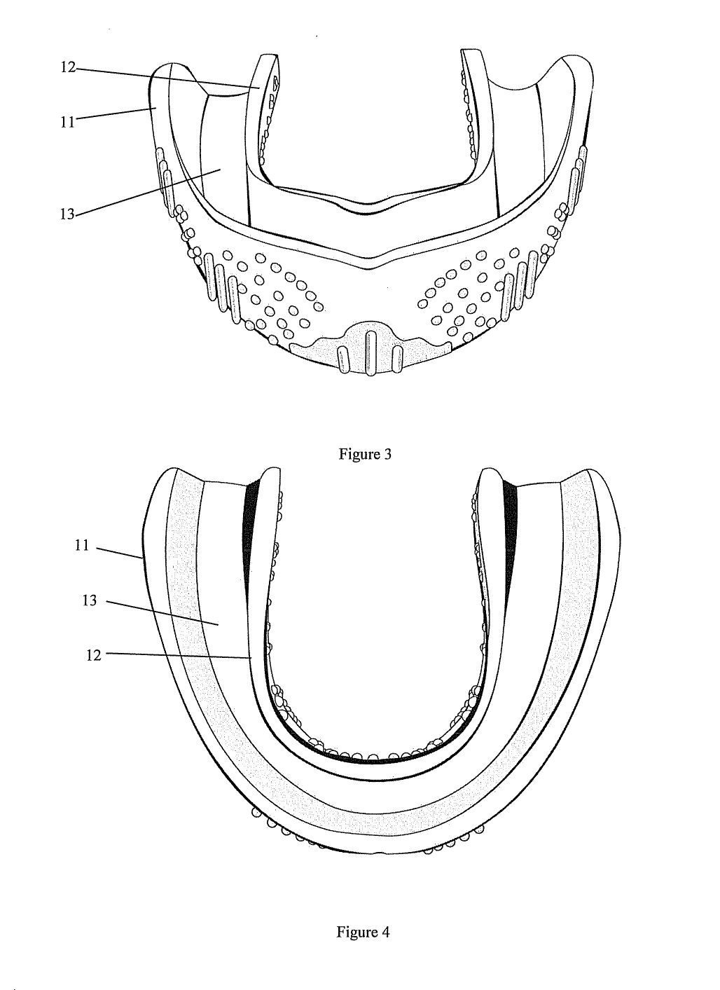

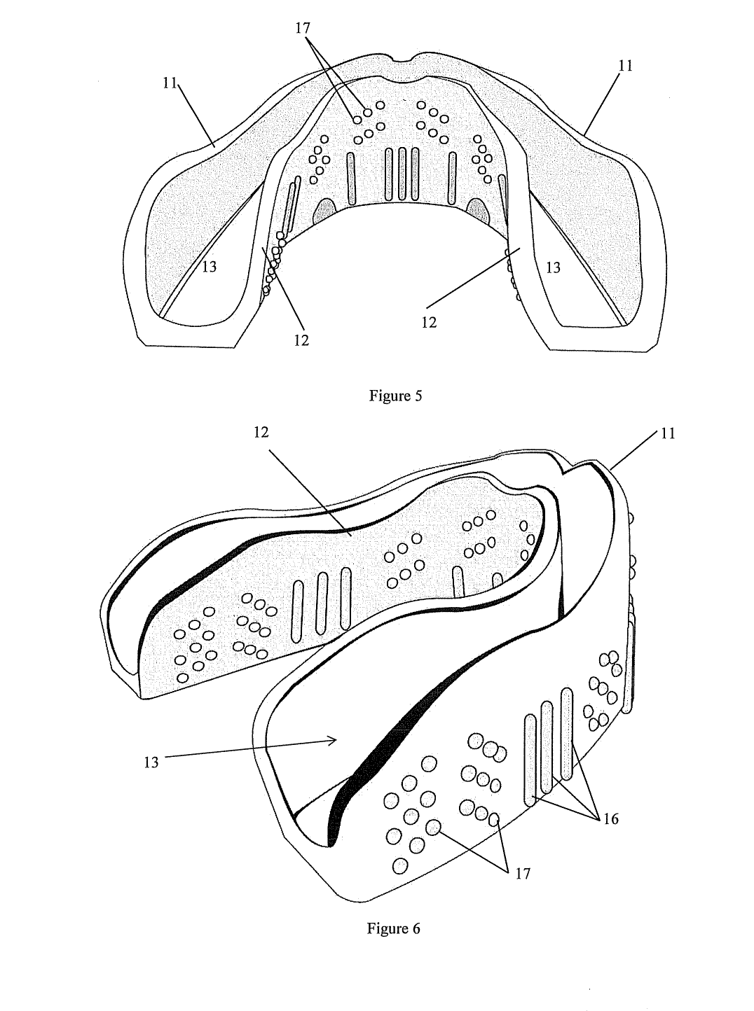

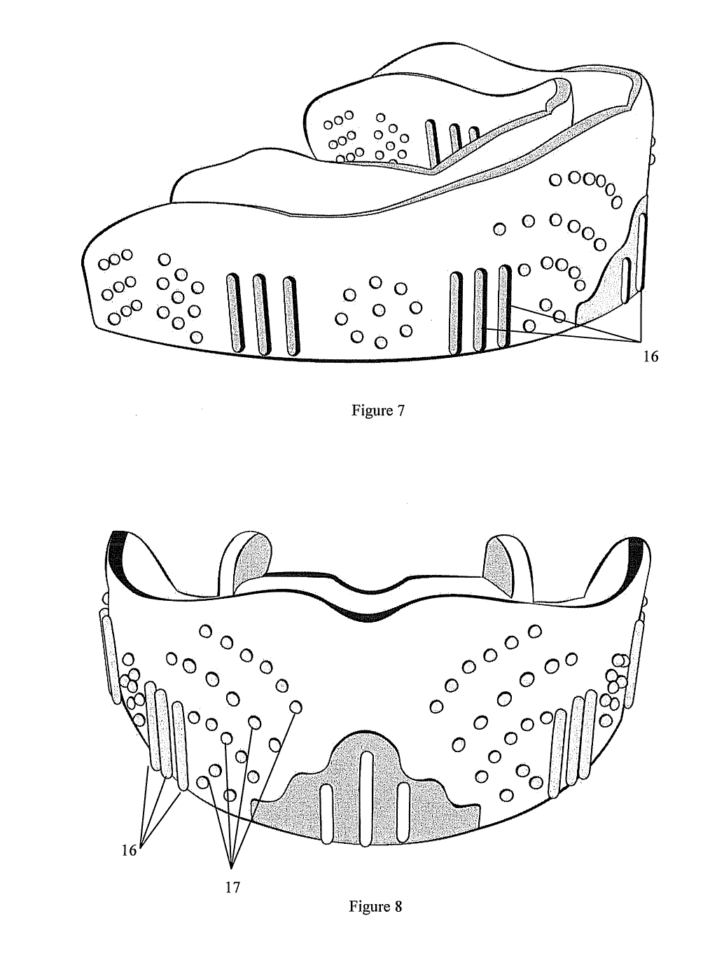

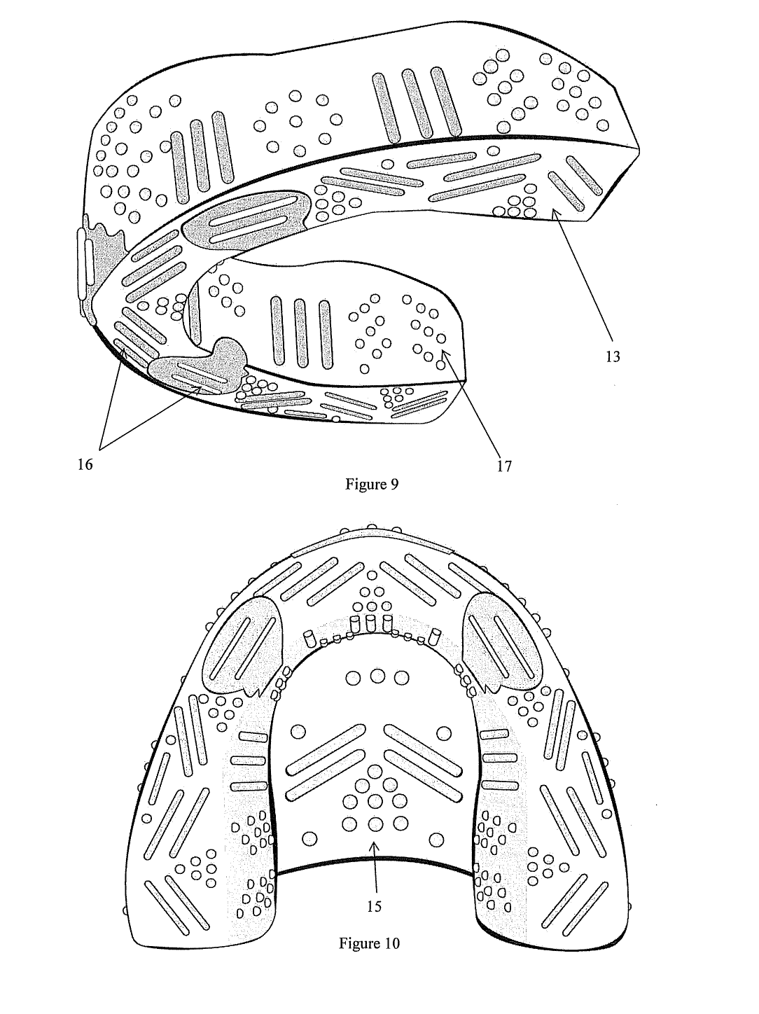

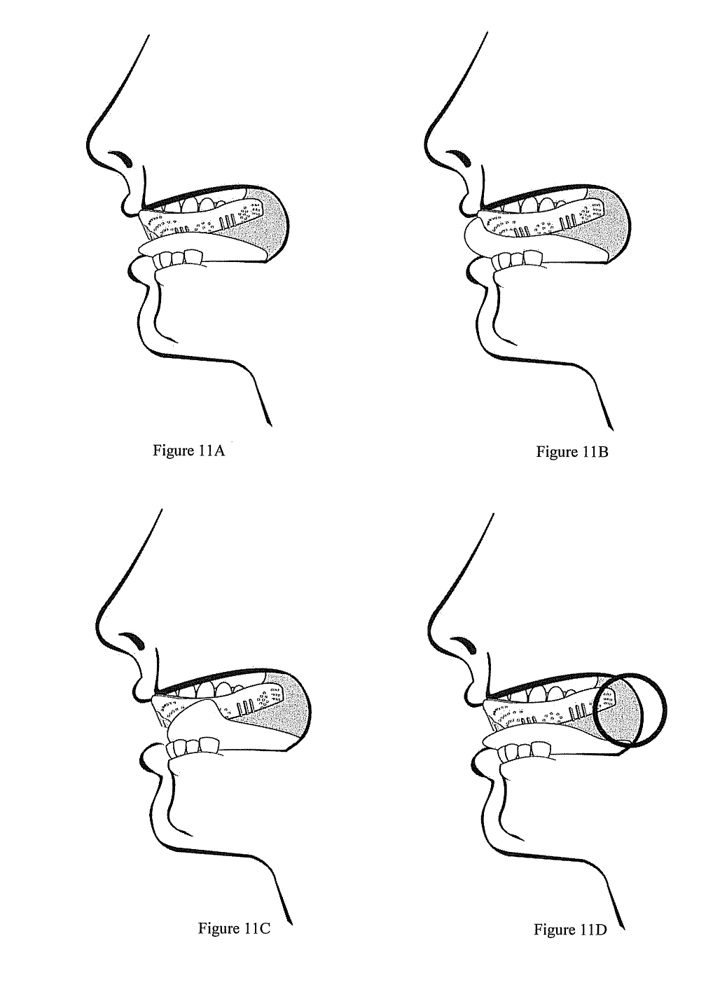

According to particularly preferred embodiments of the present invention, two configurations of tongue cleaning apparatus 10 are provided, the first embodiment illustrated in FIGS. 1 to 9 and the second embodiment illustrated in FIG. 10.

Both configurations include a positioning body moulded to fit over at least some teeth of a user and associated with at least one portion having with a plurality of shaped protrusions protruding from at least one external surface thereof. Both embodiments have a “mouthguard” configuration.

In use, the tongue cleaning apparatus is positioned within the mouth manually by a user and located adjacent the upper dental arch. The user can then press the apparatus into place. The user then moves their tongue relative to the apparatus in multiple directions and over different portions of the apparatus in order to clean their tongue. Typically, the best cleaning may be achieved if a pattern is followed. This typically utilizes the natural movements of the user's mouth and tongue to achieve cleaning of the tongue.

The tongue cleaning apparatus of the invention includes at least one positioning body molded to fit over at least some of the teeth of the user.

The most preferred positioning body is that illustrated in the Figures, namely, a generally arch shaped or U-shaped body that extends over the entire dental arch of the user. As illustrated, the body extends from the rearmost molar on one side of a user's dental arch to the rearmost molar on the other side of the dental arch. Normally, the rear of each side of the body open. The preferred configuration is therefore a positioning body which is U-shaped in plan and also substantially U-shaped in section.

In a particularly preferred embodiment, each positioning body includes a forward or outer protective flange 11 and a rearward or inner protective flange 12 connected by a protective web 13 adapted to abut or overlie the occlusal surface of at least some of the teeth in the arch. This configuration preferably defines a substantially U-shaped channel for receiving the teeth therein.

Typically, the forward or outer protective flange is higher than the inner or rearward flange.

According to the embodiment illustrated in FIG. 10, a bridge portion 14 is provided between portions of the positioning body. In use, the bridge extends over the palate of the user between lateral portions of the positioning body.

The embodiment illustrated in FIG. 10 includes a bridge portion 14 provided between the U-shaped positioning body with a rear edge 15 of the bridge portion 14 located forwardly of the rear of the positioning body. In this configuration, the rear edge 15 of the bridge portion 14 will be located approximately at the hard palate/soft palate transition or approximately ⅔ of the distance from the front of the mouth towards the throat.

The positioning body will typically be manufactured of an appropriate material for oral use. Normally, a plastic material is used and increased comfort can be provided by using rubberized or resilient plastic materials. Further, the positioning body may be formed from more than one material in order to provide the desired properties to the positioning body or at particular locations on the positioning body such as a more rigid structure in some parts with a more resilient structure in other parts.

The positioning body has an external surface having a plurality of shaped protrusions protruding therefrom.

As illustrated, the plurality of shaped protrusions are spread over the surface of the preferred arch shaped positioning body and/or bridge portion.

According to both embodiments illustrated, different types of protrusions are provided.

A first type of protrusions are elongate in the form of ridges. The elongate protrusions 16 are provided in different orientations in order to provide more effective cleaning.

The elongate protrusions will be provided with rounded ends. The preferred shape for the elongate protrusions is a rounded shape in cross-section also. The elongate protrusions can be provided in a variety of heights and lengths.

The second preferred type of protrusions are at least partially spheroidal protrusions. The spheroidal protrusions 17 are provided in discrete regions, normally in groups. Spheroidal protrusions may be provided in particular areas on the apparatus or in lines.

In the present specification and claims (if any), the word ‘comprising’ and its derivatives including ‘comprises’ and ‘comprise’ include each of the stated integers but does not exclude the inclusion of one or more further integers.

Reference throughout this specification to ‘one embodiment’ or ‘an embodiment’ means that a particular feature, structure, or characteristic described in connection with the embodiment is included in at least one embodiment of the present invention. Thus, the appearance of the phrases ‘in one embodiment’ or ‘in an embodiment’ in various places throughout this specification are not necessarily all referring to the same embodiment. Furthermore, the particular features, structures, or characteristics may be combined in any suitable manner in one or more combinations.

In compliance with the statute, the invention has been described in language more or less specific to structural or methodical features. It is to be understood that the invention is not limited to specific features shown or described since the means herein described comprises preferred forms of putting the invention into effect. The invention is, therefore, claimed in any of its forms or modifications within the proper scope of the appended claims (if any) appropriately interpreted by those skilled in the art.

What is claimed is:

1. A tongue cleaning apparatus including at least one positioning body moulded to fit over at least some teeth of a user and associated with at least one portion having with a plurality of shaped protrusions protruding from at least one external surface thereof.

2. A tongue cleaning apparatus as claimed in claim 1 wherein the at least one positioning body locates the apparatus within the user's mouth, at least temporarily, and braces the apparatus against movement which can occur due to movement of the user's tongue relative to the apparatus.

3. A tongue cleaning apparatus as claimed in claim 1 wherein the positioning body extends from a rearmost molar on one side of a user's dental arch to a rearmost molar on the other side of the dental arch.

4. A tongue cleaning apparatus as claimed in claim 1 wherein the positioning body is a U-shaped body that extends over an entire dental arch of the user.

5. A tongue cleaning apparatus as claimed in claim 4 wherein a rear portion of each side of the positioning body is open.

6. A tongue cleaning apparatus as claimed in claim 1 wherein the positioning body is located relative to an upper, superior or maxillary dental arch.

7. A tongue cleaning apparatus as claimed in claim 1 wherein the positioning body includes an outer protective flange and an inner protective flange connected by a protective web adapted to abut or overlie an occlusal surface of at least some of the teeth in the arch.

8. A tongue cleaning apparatus as claimed in claim 7 wherein the outer protective flange is higher than the protective flange.

9. A tongue cleaning apparatus as claimed in claim 7 wherein the inner and outer protective flanges are shaped to correspond to gum lines of a user.

10. A tongue cleaning apparatus as claimed in claim 1 wherein a bridge portion is provided between portions of the positioning body extending at least partially over a user's palate between lateral portions of the positioning body.

11. A tongue cleaning apparatus as claimed in claim 1 wherein the positioning body is molded to suit an individual's teeth or arch pattern in order to provide more secure engagement.

12. A tongue cleaning apparatus as claimed in claim 1 wherein the at least one portion having the shaped protrusions is an external surface of the positioning body.

13. A tongue cleaning apparatus as claimed in claim 1 wherein the plurality of shaped protrusions are spread over the positioning body.

14. A tongue cleaning apparatus as claimed in claim 1 wherein at least some of the shaped protrusions provided are elongate in the form of ridges.

15. A tongue cleaning apparatus as claimed in claim 14 wherein the elongate protrusions are provided at an angle to one another.

16. A tongue cleaning apparatus as claimed in claim 1 wherein at least some of the shaped protrusions provided are partially spheroidal protrusions.

17. A tongue cleaning apparatus as claimed in claim 16 wherein the spheroidal protrusions are provided in groups separated from one another.

18. A method for using a tongue cleaning apparatus including a positioning body moulded to fit over at least some teeth of a user and associated with at least one portion having with a plurality of shaped protrusions protruding from at least one external surface thereof, the method including the steps of fitting the body of the apparatus over at least some of teeth of a user to secure the apparatus within a user's mouth and the user moving their tongue over the apparatus to clean the user's tongue.

19. A method for using a tongue cleaning apparatus as claimed in claim 18 including the step of positioning the apparatus within the mouth located adjacent an upper arch.

20. A method for using a tongue cleaning apparatus as claimed in claim 18 wherein a pattern is followed.
